(19) United States
(12) Patent Application Publication
(10) Pub. No.: US 2007/0042375 A1
(43) Pub. Date: Feb. 22, 2007

(54) Isolated berovin photoprotein and use thereof

(75) Inventors: Stefan Golz, Essen (DE); Svetlana Markova, Krasnoyarsk (RU); Ludmila Burakova, Sosnovoborsk (RU); Ludmila Frank, Krasnoyarsk (RU); Eugene Vysotski, Krasnoyarsk (RU)

Correspondence Address: BANNER & WITCOFF, 1001 G STREET N W, SUITE 1100, WASHINGTON, DC, 20001, US

(73) Assignee: BAYER HEALTHCARE AG, Leverkusen (DE)

(21) Appl. No.: 10/569,808

(22) PCT Filed: Aug. 13, 2004
(86) PCT No.: PCT/EP04/09118

§ 371 Date: May 18, 2006

(30) Foreign Application Priority Data
Aug. 26, 2003 (DE) 103395679

(51) Int. Cl. C12Q 1/68; C07H 21/04; C12P 21/06; C07K 14/435

(52) U.S. Cl. 435/6; 435/69.1; 435/320.1; 435/325; 530/350; 536/23.5

(57) ABSTRACT

The invention relates to the photoprotein berovin, to its nucleotide and amino acid sequences and to the activity and use of the photoprotein berovin.

The invention relates to the photoprotein berovin, to its nucleotide and amino acid sequences and to the activity and use of the photoprotein berovin.

Photoproteins

The phenomenon of the generation of light by living organisms is designated bioluminescence. It is the result of biochemical reactions in cells, in which reactions the chemical energy is emitted in the form of light quanta (what is termed cold emission by means of chemoluminescence). While the light which is produced in this way is monochromatic, since it is emitted in connection with a discrete electron transfer, it can be shifted by secondary luminescent dyes (e.g. fluorescent proteins in the case of luminescent jellyfish of the genus Aequoria) into spectral regions of longer wavelength.

Bioluminescence has a diversity of biological functions: at an ocean depth of between 200 and 1000 m (mesopelagial), about 90% of all living organisms luminesce. In this case, the luminescent signals are employed for attracting partners, for deception and as a lure. Glowworms and fireflies also use the light signals for seeking partners. On the other hand, the significance of the luminescence of bacteria, fungi and single-cell algae is unclear. It is assumed that it is used for coordinating many single individuals in a large population or else represents a type of biological clock.

A large number of coelenterates are bioluminescent (Morin et al., 1974). These organisms emit blue or green light. As an isolated protein, aequorin, which is derived from Aequoria victoria (Shimomura et al., 1969) and which, in 1962, was the first light-producing protein to be identified, emitted a blue light, and not a green light as observed phenotypically in the case of Aequoria victoria. The green fluorescent protein (GFP) which, as a result of being activated by aequorin, causes Aequoria victoria to appear phenotypically green was subsequently isolated from this medusa (Johnson et al., 1962; Hastings et al., 1969; Inouye et al., 1994). Other photoproteins which have also been identified and described are clytin (Inouye et al., 1993), mitrocomin (Fagan et al., 1993) and obelin (Illarionov et al., 1995).

TABLE 1
Overview of some photoproteins. The table gives the name, the
organism from which the protein has been isolated and the identification
number (Acc. No.) of the database entry.

| Name | Organism | Identification No. |
|---|---|---|
| Obelin | Obelia geniculata | AAL86372 |
| Clytin | Clytia gregaria | CAA49754 |
| Aequorin | Aequorea macrodactyla | AAK02061 |
| Aequorin | Aequorea parva | AAK02060 |
| Mitrocomin | Mitrocoma cellularia | AAA29298 |
| Pholasin | Pholas dactylus | AAM18085 |
| | Symplectoteuthis oualaniensis | AX305029 |

TABLE 2
Overview of some photoproteins. The table gives the organism
from which the protein has been isolated, the name of the photoprotein
and a selection of patents or applications.

| | Fluorescent | |
|---|---|---|
| Organism | protein | Patent/Application |
| Obelia geniculata | Obelin | WO03006497 |
| Clytia gregaria | Clytin | WO03006497 |
| Aequoria victoria | Aequorin | WO200168824 |
| | | U.S. Pat. No. -0908909 |
| | | U.S. Pat. No. 6,152,358 |
| | | JP-0176125 |
| Pholas dactylus | Pholasin | WO0028025 |
| | | GB-0024357 |

Bioluminescence is nowadays used in technology in a wide variety of ways, e.g. in the form of bioindicators of environmental pollution or in biochemistry for sensitively detecting proteins or for quantifying particular compounds, or as what are termed reporters in connection with investigating gene regulation in the cell.

The photoproteins differ not only in their nucleotide and amino acid sequences but also in their biochemical and physical properties.

It has been demonstrated that the physical and biochemical properties of photoproteins can be altered by altering the amino acid sequences of these proteins. Examples of mutagenized photoproteins are described in the literature (U.S. Pat. No. 6,495,355; U.S. Pat. No. 5,541,309; U.S. Pat. No. 5,093,240; Shimomura et al., 1986).

The abovementioned photoproteins generate light by oxidizing coelenterazine (Haddock et al., 2001; Jones et al., 1999).

Reporter Systems

In general, genes whose gene products can be readily detected using simple biochemical or histochemical methods are termed reporter genes or indicator genes. At least 2 types of reporter gene are distinguished.
1. Resistance genes. This is the term used for genes whose expression confers, on a cell, resistance to antibiotics or other substances whose presence in the growth medium leads to the death of the cell if the resistance gene is absent.
2. Reporter genes. The products of reporter genes are used in genetic manipulation as fused or unfused indicators. The commonest reporter genes include beta-galactosidase (Alam et al., 1990), alkaline phosphatase (Yang et al., 1997; Cullen et al., 1992), and luciferases and other photoproteins (Shinomura, 1985; Phillips GN, 1997; Snowdowne et al., 1984).

The emission of photons in the visible spectral range, with this emission being effected by means of excited emitter molecules, is termed luminescence. In contrast to fluorescence, the energy is not, in this case, supplied from the exterior in the form of radiation of shorter wavelength.

A distinction is made between chemoluminescence and bioluminescence. A chemical reaction which leads to an excited molecule which itself luminesces when the excited electrons return to the basal state is termed chemoluminescence. If this reaction is catalyzed by an enzyme, the phenomenon is then referred to as being bioluminescence. The enzymes involved in the reaction are generally termed luciferases.

Classification of the Species Beroe abyssicola

Eumetazoa→Ctenophora→Cyclocoela→Beroida→Beroe abyssicola

The species Beroe abyssicola belongs to the Cnidaria, specifically to the Medusae.

Isolating the cDNA

In order to investigate the bioluminescence activity of the species, Beroe abyssicola specimens were caught in the White Sea (Kartesh Biological Station, Russia) and stored in liquid nitrogen. In order to construct the Beroe abyssicola cDNA libraries, the poly(a)+ RNA was isolated using the “Straight A” isolation method from Novagen (USA).

An RT-PCR was carried out for preparing the cDNA. For this, 1 μg of RNA was incubated with reverse transcriptase (Superscript Gold II) in accordance with the following scheme:

| | | | |
|---|---|---|---|
| PCR | 1. | 30 seconds | 95° C. |
| | 2. | 6 minutes | 68° C. |
| | 3. | 10 seconds | 95° C. |
| | 4. | 6 minutes | 68° C. |

17 cycles of step 4 after step 3

The reaction products were incubated with proteinase K, at 37° C. for 30 minutes, in order to inactivate the polymerase, and the cDNA was precipitated with ethanol. The cDNA expression library was constructed using the Clontech (USA) “SMART cDNA” library construction kit in accordance with the manufacturer's instruction. The cDNA was cloned into the expression vector pTriplEx2 (Clontech; USA). The expression vectors were transformed by electroporation into bacteria of the strain E. coli XL1 blue.

The bacteria were plated out on solid LB nutrient medium and incubated at 37° C. for 24 hours. A replica plating was then carried out, with the bacteria being transferred to another solid nutrient medium plate using a nitrocellulose filter. The replica plate was in turn incubated at 37° C. for 24 hours and the bacterial colonies which had grown were transferred into liquid LB medium. After IPTG (final concentration, 0.1 mM) had been added, the bacteria were incubated at 37° C. for 4 hours on a shaker. The bacteria were harvested by centrifugation and the bacterial mass was resuspended, at 0° C., in 0.5 ml of disruption buffer (5 mM EDTA, 20 mM Tris-HCL, pH 9.0). The bacteria were then disrupted by ultrasonication.

After adding coelenterazine (final concentration, 10E-07 M), the lysates were incubated at 4° C. for 3 hours. The bioluminescence was then measured in a luminometer after adding calcium chloride (final concentration, 20 mM).

A photoprotein was identified. The photoprotein was designated berovin. The photoprotein berovin is described in detail below.

Berovin

With an identity of 29%, the photoprotein berovin exhibits the highest homology at the amino acid level with obelin from Obelia longissima (shown in Example 6). At the nucleic acid level, the identity is less than 30% (shown in Example 6). The BLAST method (Altschul et al., 1997) was used for the sequence comparison.

The bioluminescent properties of Beroe species have already been described phenomenologically before (Ward et al., 1974; Ward et al., 1975).

The invention also relates to functional equivalents of berovin. Functional equivalents are those proteins which have comparable physicochemical properties and are at least 70% homologous with SEQ ID NO: 2. Preference is given to a homology of at least 80% or 90%. A homology of at least 95% is particularly preferred.

The photoprotein berovin is suitable for being used as a reporter gene for cellular systems, especially for receptors, for ion channels, for transporters, for transcription factors or for inducible systems.

The photoprotein berovin is suitable for being used as a reporter gene in bacterial and eukaryotic systems, especially in mammalian cells, in bacteria, in yeasts, in baculo and in plants.

The photoprotein berovin is suitable for being used as a reporter gene for cellular systems in combination with bioluminescent or chemoluminescent systems, especially systems using luciferases, using oxygenases or using phosphatases.

The photoprotein berovin is suitable for being used as a fusion protein, especially for receptors, for ion channels, for transporters, for transcription factors, for proteinases, for kinases, for phosphodiesterases, for hydrolases, for peptidases, for transferases, for membrane proteins and for glycoproteins.

The photoprotein berovin is suitable for being immobilized, especially by antibodies, by biotin, or by magnetic or magnetizable supports.

The photoprotein berovin is suitable for being used as a protein for energy transfer systems, especially FRET (fluorescence resonance energy transfer), BRET (bioluminescence resonance energy transfer), FET (field effect transistors), FP (fluorescence polarization) and HTRF (homogeneous time-resolved fluorescence) systems.

The photoprotein berovin is suitable for labeling substrates or ligands, especially for proteases, for kinases or for transferases.

The photoprotein berovin is suitable for being expressed in bacterial systems, especially for titer determination, as a substrate for biochemical systems, especially for proteinases and kinases.

The photoprotein berovin is suitable for being used as a label, especially coupled to antibiotics, coupled to enzymes, coupled to receptors or coupled to ion channels and other proteins.

The photoprotein berovin is suitable for being used as a reporter gene in the search for pharmacological active compounds, especially in HTS (high throughput screening).

The photoprotein berovin is suitable for being used as a component of detection systems, especially for ELISA (enzyme-linked immunosorbent assay), for immunohistochemistry, for Western blotting or for confocal microscopy.

The photoprotein berovin is suitable for being used as a label for analyzing interactions, especially for protein-protein interactions, for DNA-protein interactions, for DNA-RNA interactions, for RNA-RNA interactions, or for RNA-protein interactions (DNA: desoxyribonucleic acid; RNA: ribonucleic acid).

The photoprotein berovin is suitable for being used as a label or fusion protein for expression in transgenic organisms, especially in mice, in rats, in hamsters and other mammals, in primates, in fish, in worms or in plants.

The photoprotein berovin is suitable for being used as a label or fusion protein for analyzing embryonic development.

The photoprotein berovin is suitable for being used as a label by way of a coupling mediator, especially by way of biotin, by way of NHS (N-hydroxysulfosuccimide) or by way of CN—Br.

The photoprotein berovin is suitable for being used as a reporter which is coupled to nucleic acids, especially to DNA or RNA.

The photoprotein berovin is suitable for being used as a reporter which is coupled to proteins or peptides.

The photoprotein berovin is suitable for being used as a reporter for measuring intracellular or extracellular calcium concentrations.

The photoprotein berovin is suitable for characterizing signal cascades in cellular systems.

The photoprotein berovin which is coupled to nucleic acids or peptides is suitable for being used as a probe, especially for Northern blots, for Southern blots, for Western blots, for ELISA, for nucleic acid sequencings, for protein analyses or for chip analyses.

The photoprotein berovin is suitable for being used for labeling pharmacological formulations, especially infectious agents, antibodies or “small molecules”.

The photoprotein berovin is suitable for being used for geological investigations, especially for ocean, groundwater and river currents.

The photoprotein berovin is suitable for being expressed in expression systems, especially in in-vitro translation systems, in bacterial systems, in yeast systems, in baculo systems, in viral systems and in eukaryotic systems.

The photoprotein berovin is suitable for visualizing tissues or cells in connection with surgical interventions, especially in connection with invasive, in connection with noninvasive and in connection with minimally invasive interventions.

The photoprotein berovin is also suitable for labeling tumor tissues and other phenotypically altered tissues, especially in connection with histological investigation and in connection with surgical interventions.

The invention also relates to the purification of the photoprotein berovin, especially as a wild-type protein, as a fusion protein and as a mutagenized protein.

The invention also relates to the use of the photoprotein berovin in the field of cosmetics, especially bath additives, lotions, soaps, body dyes, toothpaste and body powders.

The invention also relates to the use of the photoprotein berovin for dyeing, especially dyeing foodstuffs, bath additives, ink, textiles and plastics.

The invention also relates to the use of the photoprotein berovin for dyeing paper, especially greetings cards, paper products, wallpapers and handicraft articles.

The invention also relates to the use of the photoprotein berovin for dyeing liquids, especially for water pistols, fountains, beverages and ice.

The invention also relates to the use of the photoprotein berovin for producing toys, especially finger dye and makeup.

The invention relates to nucleic acid molecules which encode the polypeptide which is disclosed by SEQ ID NO: 2.

The invention relates to the polypeptide having the amino acid sequence which is disclosed in SEQ ID NO: 2.

The invention furthermore relates to nucleic acid molecules which are selected from the group consisting of
a) nucleic acid molecules which encode a polypeptide which comprises the amino acid sequence disclosed by SEQ ID NO: 2;
b) nucleic acid molecules which contain the sequence depicted by SEQ ID NO: 1;
c) nucleic acid molecules whose complementary strand hybridizes with a nucleic acid molecule from a) or b) under stringent conditions and which exhibit the biological function of a photoprotein;
   A stringent hybridization of nucleic acid molecules can be carried out, for example, in an aqueous solution comprising 0.2×SSC (1× standard saline citrate=150 mM NaCl, 15 mM trisodium citrate) at 68° C. (Sambrook et al., 1989).
d) nucleic acid molecules which differ from the nucleic acid molecules mentioned under c) due to the degeneracy of the genetic code;
e) nucleic acid molecules which exhibit a sequence homology with SEQ ID NO: 1 of at least 95% and whose protein product exhibits the biological function of a photoprotein; and
f) nucleic acid molecules which exhibit a sequence homology with SEQ ID NO: 1 of at least 65% and whose protein product exhibits the biological function of a photoprotein.

The invention also relates to nucleic acid molecules which exhibit a sequence homology with SEQ ID NO: 1 of at least 95%, 90%, 85%, 80%, 75%, 70%, 65% or 60% and which encode a polypeptide which possesses the properties of a photoprotein.

The invention relates to the abovementioned nucleic acid molecules in which the sequence contains a functional promoter 5′ to the photoprotein-encoding sequence.

The invention also relates to nucleic acid molecules as previously described which are constituents of recombinant DNA or RNA vectors.

The invention relates to organisms which harbor such a vector.

The invention relates to oligonucleotides having more than 10 consecutive nucleotides which are identical or complementary to the DNA or RNA sequence of the berovin molecules or of the other molecules according to the invention.

The invention relates to photoproteins which are encoded by the previously described nucleotide sequences.

The invention relates to methods for expressing the photoprotein polypeptides according to the invention in bacteria, in eukaryotic cells or in in-vitro expression systems.

The invention also relates to methods for purifying/isolating a photoprotein polypeptide according to the invention.

The invention relates to peptides which have more than 5 consecutive amino acids and which are immunologically recognized by antibodies directed against the photoproteins according to the invention.

The invention relates to the use of the photoprotein-encoding nucleic acids according to the invention as marker genes or reporter genes, in particular for searching for pharmacological active compounds and for diagnostics.

The invention relates to the use of the photoproteins according to the invention or of a photoprotein-encoding nucleic acid according to the invention as labels or reporters or as a marker gene or reporter gene.

The invention relates to the use of the photoprotein berovin (SEQ ID NO: 2), or to the use of a nucleic acid which encodes the photoprotein berovin as a label or reporter, or as a label or reporter gene, in particular for searching for pharmacological active compounds and for diagnostics.

The invention relates to the use of the nucleic acid depicted in SEQ ID NO: 1 as a marker gene or reporter gene, in particular for searching for pharmacological active compounds and diagnostics.

The invention relates to the use of the peptide depicted in SEQ ID NO: 3 and its underlying nucleic acid sequence SEQ ID NO: 1 as a marker gene or reporter gene, in particular for searching for pharmacological active compounds and diagnostics.

The invention also relates to polyclonal or monoclonal antibodies which recognize a polypeptide according to the invention.

The invention also relates to monoclonal or polyclonal antibodies which recognize the photoprotein berovin (SEQ ID NO: 2).

Expressing the Photoproteins of the Invention

The production of a molecule which, after the gene has been introduced into a suitable host cell, enables the foreign gene which is cloned into an expression vector to be transcribed and translated is termed expression. Expression vectors contain the control signals which are required for expressing genes in prokaryotic or eukaryotic cells.

In principle, expression vectors can be constructed in two different ways. In the case of what are termed transcription fusions, the protein encoded by the cloned-in foreign gene is synthesized as an authentic, biologically active protein. For this purpose, the expression vector carries all the 5′ and 3′ control signals which are required for the expression.

In the case of what are termed translation fusions, the protein encoded by the cloned-in foreign gene is expressed, together with another protein which can be detected readily, as a hybrid protein. The 5′ and 3′ control signals which are required for the expression, including the start codon and, possibly, a part of the sequences encoding the N-terminal regions of the hybrid protein to be formed, originate from the vector. The additionally inserted protein moiety not only in many cases stabilizes the protein, which is encoded by the cloned-in foreign gene, against breakdown by cellular proteases; it can also be used for detecting and isolating the hybrid protein which is formed. The expression can take place either transiently or stably. Suitable host organisms are bacteria, yeasts, viruses or eukaryotic systems.

Purifying the Photoproteins of the Invention

The isolation of proteins (after they have been overexpressed as well) is frequently termed protein purification. A large number of established methods are available for purifying proteins.

The solid/liquid separation is a basic operation in connection with isolating proteins. This procedural step is required when separating cells from the culture medium, when clarifying the crude extract after having disrupted the cells and removing the cell debris, and when separating off sediments after precipitations, etc. It takes place by means of centrifugation and filtration.

In order to obtain intracellular proteins, the cell wall must be destroyed or rendered permeable. High-pressure homogenizers or agitator ball mills or glass bead mills are used for this purpose, depending on the scale and the organism. Mechanical cell integrations and ultrasonic treatment are used, inter alia, on the laboratory scale.

Both in the case of extracellular proteins and in the case of intracellular proteins (following cell disruption), various precipitation methods using salts (in particular ammonium sulfate) or organic solvents (alcohols or acetone) represent rapid and efficient methods for concentrating proteins. When intracellular proteins are being purified, it is desirable to remove the soluble nucleic acids (precipitation with, for example, streptomycin sulfate or protamine sulfate). When extracellular proteins are being isolated, carriers (e.g. starch or kieselguhr) are frequently added before adding the precipitating agents in order to obtain sediments which are easier to handle.

Numerous chromatographic methods and partition methods (absorption chromatography and ion exchange chromatography, gel filtration, affinity chromatography and electrophoreses) are available for high-degree purification. Column chromatography is also used on an industrial scale. Affinity chromatography, which makes possible purification factors of up to several 100s per step, is especially important for the laboratory scale.

Extracellular proteins accrue in relatively dilute solutions. Just like extracellular proteins, they have to be concentrated before being subjected to further use. In addition to the methods which have already been mentioned, ultrafiltration has proved to be of value, on an industrial scale as well.

Inorganic salts which accompany proteins are frequently undesirable in the case of specific applications. They can be removed by, inter alia, gel filtration, dialysis and diafiltration.

A large number of proteins are used as dry preparations. Important drying methods are vacuum drying, freeze drying and spray drying.

Nucleotide and Amino Acid Sequences

The photoprotein berovin is encoded by the
following nucleotide sequence (SEQ ID NO: 1):
5′-GAGTTTTAAACTTTATACAACACTACTTTATAAAATCTAATTTGAGC
CAATCAAAATGACTGAACGTCTGAACGAGCAGAACAACGAGAGTTACCGC
TACCTGAGAAGCGTGGGAAACCAGTGGCAGTTCAACGTAGAGGACCTCCA
CCCCAAGATGTTGTCCCGTCTCTACAAGAGATTCGATACTTTCGATCTAG
ACAGTGACGGTAAGATGGAGATGGACGAGGTCTTGTACTGGCCCGACAGG
ATGAGGCAGCTGGTAAACGCTACTGATGAGCAGGTTGAGAAGATGCGGGA
TGCTGTGAGAGTTTTCTTTTTGCACAAGGGAGTGGAGCCAGTAAACGGTC
TCCTCAGAGAGGACTGGGTGGAAGCTAACAGAGTCTTCGCTGAGGCTGAG
AGAGAAAGAGAGCGACGAGGAGAACCTTCTCTTATCGCACTTCTCTCCAA
CTCTTACTACGATGTACTGGATGATGACGGTGATGGTACTGTTGACGTCG
ATGAATTAAAGACCATGATGAAAGCATTTGATGTGCCCCAGGAAGCTGCC
TACACCTTCTTCGAGAAGGCAGACACTGACAAGAGTGGAAAGTTGGAGAG
AACAGAACTAGTTCATCTCTTTAGAAAGTTTTGGATGGAGCCTTACGATC
CACAGTGGGACGGAGTCTACGCTTATAAGTACTAATAAATTATCGGATCC
AGAACAAACGGCAAGAACTATTTTACACTCCACTTCAATTATAAACGATG
ATTTCATCGTTTCATGAAAACAAATTTTAGCATAAAACAATAAACATTTT
CGACTACTAAAATTACAGTCAACATAAAAATTTTAAAGTGTGTGATAAAT
TTTTCTGAATGTCTCCTAACTTCGATAATAATCTTCAAACTTTTAGTCAA
TATATGGAAATAAAAATATTTTGGTTTGATGAGATGATAAAACTTTTGTT
TTAAATTTTAGTTGGAGTAATTCATTGAATATGAAATGGACTTCGGACAA
TTACCCTGGCCTTCTGTATATTTAAAGCATGCTTTCCGTCAATAATTTGT
TGTATCTATGTATTTATTTGTACATTAATTTAACCATATAGAATTATCTG
TATAATCCCTGTTATATTTATACACTGCATTACAAAAAAAAAAAAAAAAA
AAAAAAAAAAAAA-3′
This yields an amino acid sequence of (SEQ ID NO:
2):
MTERLNEQNNESYRYLRSVGNQWQFNVEDLHPKMLSRLYKRFDTFDLDSD
GKMEMDEVLYWPDRMRQLVNATDEQVEKMRDAVRVFFLHKGVEPVNGLLR
EDWVEANRVFAEAERERERRGEPSLIALLSNSYYDVLDDDGDGTVDVDEL
KTMMKAFDVPQEAAYTFFEKADTDKSGKLERTELVHLFRKFWMEPYDPQW
DGVYAYKY

These sequences are reproduced in the sequence listing.

### BRIEF DESCRIPTION OF THE FIGURES

FIG. 1: FIG. 1 shows the plasmid map of the vector pTriplEX2-berovin.

FIG. 2: FIG. 2 shows the plasmid map of the vector pcDNA3-berovin.

FIG. 3: FIG. 3 shows the bioluminescence activity of berovin following bacterial expression. (Y=RLU: relative light units; X=dilution; black bar=berovin; gray bar=control lysate).

FIG. 4: FIG. 4 shows the bioluminescence activity of berovin following expression in CHO cells. (Y=RLU: relative light units; X=ATP (logarithmic representation in mol/l)).

FIG. 5: FIG. 5 shows the kinetic analysis of the bioluminescence of berovin. (Y=RLU: relative light units; X=time [seconds]).

FIG. 6: FIG. 6 shows the kinetic analysis of the bioluminescence of obelin. (Y=RLU: relative light units; X=time [seconds]).

FIG. 7: FIG. 7 shows the alignment of berovin and obelin (Obelia longissima) at the amino acid level.

### EXAMPLES

### Example 1

The Clontech plasmid pTriplEx2 was used as vector for preparing the construct which is described below. The derivative of the vector was designated pTriplEx2-berovin. The vector pTriplEx2-berovin was used for expressing berovin in bacterial systems.

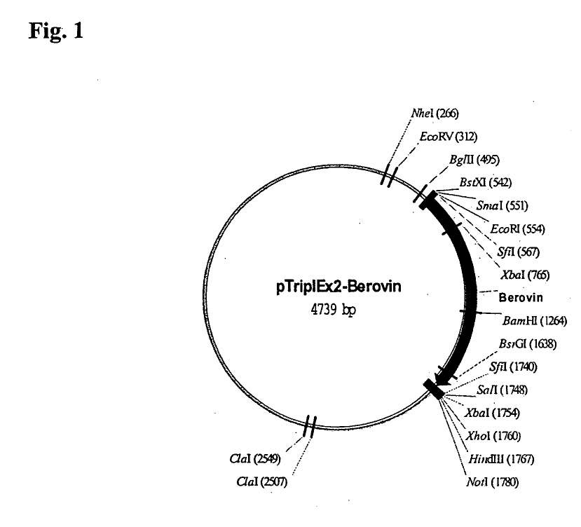

FIG. 1 shows the plasmid map of the vector pTriplEX2-berovin.

### Example 2

The Clontech plasmid pcDNA3.1(+) was used as the vector for preparing the construct which is described below. The derivative of the vector was designated pcDNA3-berovin. The vector pcDNA3-berovin was used for expressing berovin in eukaryotic systems.

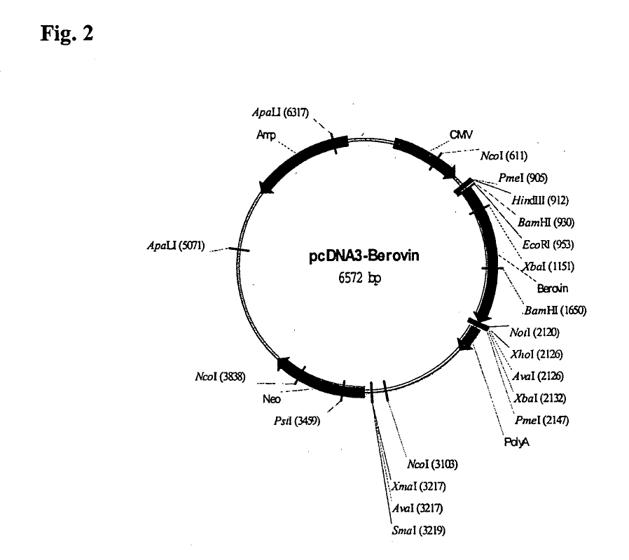

FIG. 2 shows the plasmid map of the vector pcDNA3-berovin.

### Example 3

Bacterial Expression

The bacterial expression was effected in the E. coli strain BL21 (DE3) by transforming the bacteria with the expression plasmids pTriplEX2-berovin and pTriplEX2. The transformed bacteria were incubated at 37° C. for 3 hours in LB medium and the expression was induced for 4 hours by adding IPTG up to a final concentration of 1 mM. The induced bacteria were harvested by centrifugation, resuspended in 50 mM Tris/HCl (pH 9.0)+5 mM EDTA and disrupted by ultrasonication. The lysate was subsequently centrifuged at 13 000 rpm (16 000 ref) for 15 minutes and the supernatant removed. The supernatant (dilutions 1:5, 1:10; 1:20 and 1:50 with Tris/HCl pH 9.0)) was incubated with coelenterazine (10E-07 M coelenterazine in Tris/HCl pH 9.0) for 3 hours in the dark. The bioluminescence was measured in a luminometer directly after adding 5 mM calcium chloride. The measurement integration time was 40 seconds.

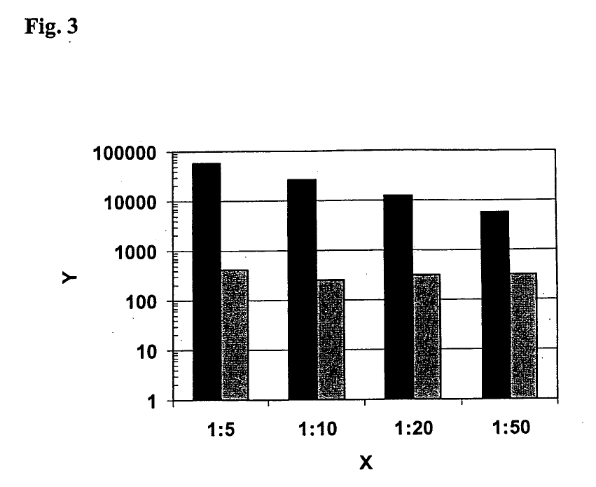

FIG. 3 shows the results of measuring the bioluminescence of berovin in bacteria.

### Example 4

Eukaryotic Expression

Constitutive eukaryotic expression was effected in CHO cells by transfecting the cells with the expression plasmids pcDNA3-berovin and pcDNA3.1(+) in transient experiments. For this, 10 000 cells per well were plated out, in DMEM-F12 medium, on 96-well microtiter plates and the plates were incubated overnight at 37° C. Transfection was effected using the Fugene 6 kits (Roche) in accordance with the manufacturer's instructions. The transfected cells were incubated overnight in DMEM-F12 medium at 37° C. The medium was then removed and replaced with 50 μl of coelenterazine (10E-07 M coelenterazine in PBS). The cells were incubated at 37° C. for 3 hours and ATP (adenosine triphosphate) was then added to a final concentration of 1 M. The measurement in a luminometer was started directly after the addition. The integration time was 1 second, with the total measurement time being 60 seconds.

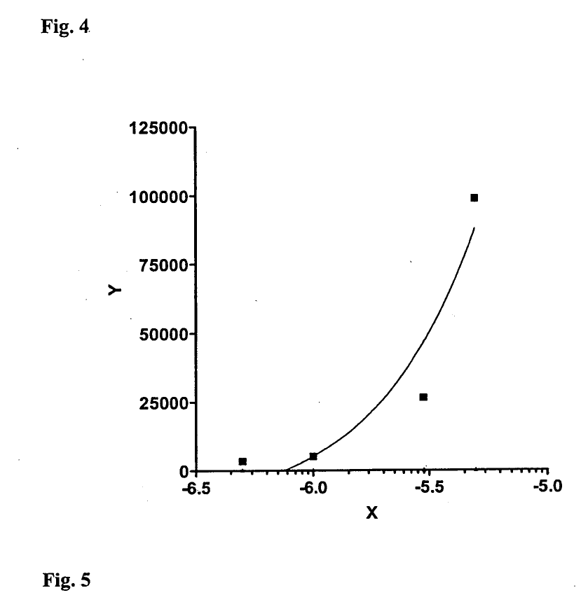

FIG. 4 shows the results of measuring the bioluminescence of berovin in CHO cells.

### Example 5

Result of a BLAST Analysis of Berovin at the Amino Acid Level:

>pdb|1JF2|A Chain A, Crystal Structure Of W92f Obelin Mutant From Obelia Longissima At 1.72 Angstrom Resolution, Length=195, Score=82.4 bits (209), Expect=4e-15, Identities=52/177 (29%), Positives=88/177 (49%), Gaps=4/177 (2%)
>emb|CAD87675.1| unnamed protein product [synthetic construct], Length=195, Score=82.0 bits (201), Expect=6e-15, Identities=52/177 (29%), Positives=88/177 (49%), Gaps=4/177 (2%)
>emb|CAD87673.1| unnamed protein product [synthetic construct], Length=195, Score=82.0 bits (201), Expect=6e-15, Identities=52/177 (29%), Positives=88/177 (49%), Gaps=4/177 (2%)
>emb|CAD87672.1| unnamed protein product [synthetic construct], Length=195, Score=82.0 bits (201), Expect=6e-15, Identities=52/177 (29%), Positives=88/177 (49%), Gaps4/177 (2%)
>emb|CAD87671.1| unnamed protein product [Obelia longissima], Length=195, Score=82.0 bits (201), Expect=6e-15, Identities=52/177 (29%), Positives=88/177 (49%), Gaps=4/177 (2%)

### Example 6

Result of a BLAST Analysis of Berovin at the Nucleic Acid Level:

>emb|AL162584.9| Human DNA sequence from clone RP11-12A16 on chromosome 9, complete sequence, Length=160178, Score=50.7 bits (26), Expect=7e-04, Identities=28/29 (96%)
>gb|BC044324.1| Xenopus laevis, clone MGC:52816 IMAGE:4724996, mRNA, complete cds, Length=2718, Score=44.9 bits (23), Expect=0.040, Identities=25/26 (96%)
>emb|AJ298151.1|XLA298151 Xenopus laevis mRNA for beta-amyloid precursor protein B (app gene), Length=2740, Score=44.9 bits (23), Expect=0.040
Identities=25/26 (96%)
>emb|AL139825.14| Human DNA sequence from clone RP11-467D7 on chromosome 20. Contains a novel gene, ESTs, STSs and GSSs, complete sequence, Length=37526, Score=44.9 bits (23), Expect=0.040, Identities=27/29 (93%)
>gb|U50135.1| Caenorhabditis elegans cosmid C52E12, complete sequence, Length=40420, Score=44.9 bits (23), Expect=0.040, Identities=25/26 (96%)
>gb|AC126802.41 Mus musculus chromosome 5 clone RP24-543J12, complete sequence, Length=321487615, Score=43.0 bits (22), Expect=0.15, Identities=30/34 (88%)

### Example 7

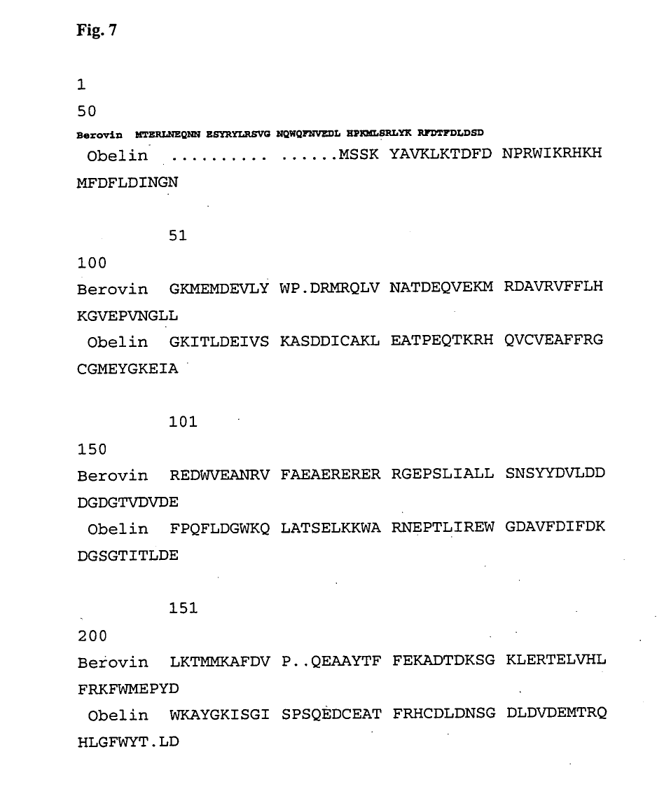

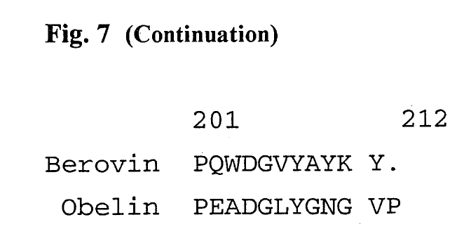

FIG. 7 shows the alignment of berovin with obelin (Obelia longissima) at the amino acid level.

### Example 8

Kinetic Analysis of Berovin

For the kinetic analysis of the bioluminescence of berovin, CHO cells were transiently transfected with pcDNA3-berovin or pcDNA-obelin or pcDNA3 (without any integrated cDNA). The transfection and measurement were carried out as described in Example 4. The readings were taken for a period of 60 seconds using an integration time of 1 second.

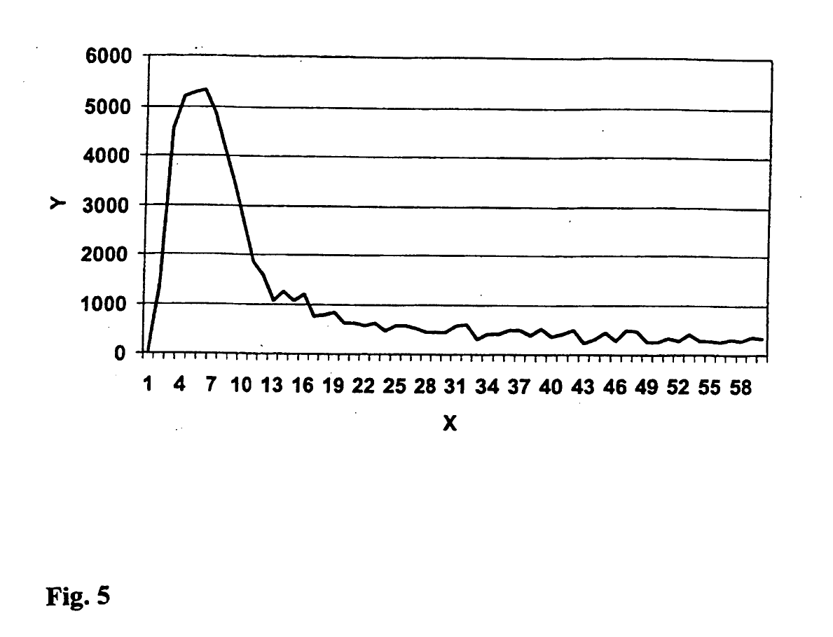

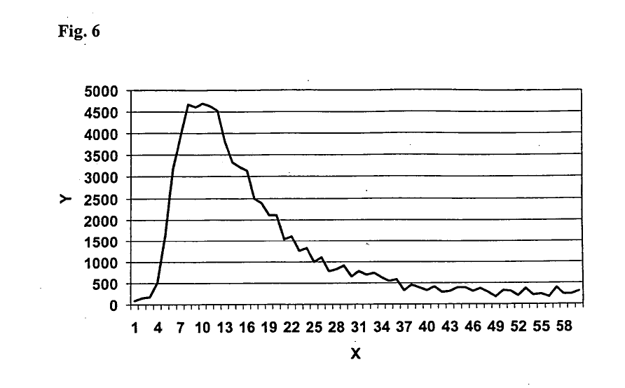

FIGS. 5 and 6 show the results of the kinetic analysis of berovin and obelin.

### LITERATURE/PATENTS

U.S. Pat. No. 6,495,355
U.S. Pat. No. 5,541,309
U.S. Pat. No. 5,093,240
U.S. Pat. No. 0,908,909
U.S. Pat. No. 6,152,358
JP-0176125
GB-0024357
WO03006497
WO200168824
Alam J, Cook J L. Reporter genes: application to the study of mammalian gene transcription. Anal Biochem. 1990 Aug. 1; 188(2):245-54
Altschul, Stephen F., Thomas L. Madden, Alejandro A. Schäffer, Jinghui Zhang, Zheng Zhang, Webb Miller, and David J. Lipman (1997); Gapped BLAST and PSI-BLAST: a new generation of protein database search programs; Nucleic Acids Res. 25:3389-3402
Chiesa A, Rapizzi E, Tosello V, Pinton P, de Virgilio M, Fogarty K E, Rizzuto R. Recombinant aequorin and green fluorescent protein as valuable tools in the study of cell signalling. Biochem J. 2001 Apr. 1; 355(Pt 1):1-12.
Claros, M. G., Vincens, P. (1996); Computational method to predict mitochondrially imported proteins and their targeting seqeunces. Eur. J. Biochem 241, 779-786.
Cullen Bryan R., Malim Michael H., Secreted placental alkaline phosphatase as a eukaryotic reporter gene. Methods in Enzymology. 216:362ff
Fagan T F, Ohmiya Y, Blinks J R, Inouye S, Tsuji F I. Cloning, expression and sequence analysis of cDNA for the Ca(2+)-binding photoprotein, mitrocomin. FEBS Lett. 1993 Nov. 1; 333(3):301-5
Hastings, J. W. and Morin, J. G. (1969) Comparative biochemistry of calcium-activated photoproteins from the ctenophore, Mnemiopsis and the coelenterates Aequorea, Obelia, and Pelagia. Biol. Bull. 137, 402.
Haddock S H, Rivers T J, Robison B H. Can coelenterates make coelenterazine? Dietary requirement for luciferin in cnidarian bioluminescence. Proc Natl Acad Sci USA 2001 Sep. 25; 98(20): 11148-51
Inouye S, Tsuji F I. (1994) Aequorea green fluorescent protein. Expression of the gene and fluorescence characteristics of the recombinant protein. FEBS Lett 1994 Mar. 21; 341(2-3):277-80
Inouye S, Tsuji F.I. Cloning and sequence analysis of cDNA for the Ca(2+)-activated photoprotein, clytin. FEBS Lett. 1993 Jan. 11; 315(3):343-6.
Illarionov B A, Bondar V S, Illarionova V A, Vysotski E S. Sequence of the cDNA encoding the Ca(2+)-activated photoprotein obelin from the hydroid polyp Obelia longissima. Gene. 1995 Feb. 14; 153(2):2734.
Jones K, Hibbert F, Keenan M. Glowing jellyfish, luminescence and a molecule called coelenterazine. Trends Biotechnol 1999 December; 17(12):477-81
Johnson, F. H., Shimomura, O., Saiga, Y., Gershman, L. C., Reynolds, G. T., and Waters, J. R. (1962) Quantum efficiency of Cypridina luminescence, with a note on that of Aequorea. J. Cell. Comp. Physiol. 60, 85-103.
Morin, J. G. and Hastings, J. W. (1971) Biochemistry of the bioluminescence of colonial hydroids and other coelenterates. J. Cell. Physiol. 77, 305-311.
Phillips G N. Structure and dynamics of green fluorescent protein. Curr Opin Struct Biol. 1997 December; 7(6):821-7
Sambrook, J., Fritsch, E. Maniatis, T. 1989, Molecular cloning. A laboratory manual Vol 1-3, Cold Spring Harbor, N.Y.: Cold Spring Harbor Laboratory Press
Shimomura O, Johnson F H. Properties of the bioluminescent protein aequorin. Biochemistry. 1969 October; 8(10):3991-7
Shimomura O., Bioluminescence in the sea: photoprotein systems. Symp Soc Exp Biol. 1985; 39:351-72
Shimomura O. Isolation and properties of various molecular forms of aequorin. Biochem J. 1986 Mar. 1; 234(2):271-7.
Snowdowne K W, Borle A B. Measurement of cytosolic free calcium in mammalian cells with aequorin. Am J Physiol. 1984 November; 247(5 Pt 1):C396-408.
Ward W W, Seliger H H. (1974) Properties of mnemiopsin and berovin, calcium-activated photoproteins from the ctenophores Mnemiopsis sp. and Beroe ovata. Biochemistry. 1974 Mar. 26; 13(7): 1500-10.
Ward W W, Cormier M J. (1975) Extraction of Renilla-type luciferin from the calcium-activated photoproteins aequorin, mnemiopsin, and berovin. Proc Natl Acad Sci USA. 1975 July; 72(7):2530-4
Yang Te-Tuan, Sinai Parisa, Kitts Paul A. Kain Seven R., Quantification of gene expression with a secreted alkaline phosphatase reporter system. Biotechnique. 1997 23(6) 1110ff

1. A nucleic acid molecule which is selected from the group consisting of
a) nucleic acid molecules which encode a polypeptide which comprises the amino acid sequence disclosed by SEQ ID NO: 2;
b) nucleic acid molecules which contain the sequence depicted by SEQ ID NO: 1;
c) nucleic acid molecules whose complementary strand hybridizes with a nucleic acid molecule from a) or b) under stringent conditions and which exhibit the biological function of a photoprotein;
d) nucleic acid molecules which differ from the nucleic acid molecules mentioned under c) due to the degeneracy of the genetic code;
e) nucleic acid molecules which exhibit a sequence homology with SEQ ID NO: 1 of at least 95% and have the biological function of a photoprotein; and
f) nucleic acid molecules which exhibit a sequence homology with SEQ ID NO: 1 of at least 65% and have the biological function of a photoprotein.

2. The nucleic acid as claimed in claim 1 which contains a functional promoter 5′ to the photoprotein-encoding sequence.

3. The nucleic acid of claim 2 which is in a recombinant DNA or RNA vector.

4. An organism comprising the vector as claimed in claim 3.

5. An oligonucleotide having more than 10 consecutive nucleotides which is identical or complementary to a constituent sequence of a nucleic acid molecule as claimed in claim 1.

6. A polypeptide which is encoded by a nucleic acid molecule selected from the group consisting of
(a) nucleic acid molecules which encode a polypeptide which comprises the amino acid sequence disclosed by SEQ ID NO: 2.
(b) nucleic acid molecules which contain the sequence depicted by SEQ ID NO: 1;
(c) nucleic acid molecules whose complementary strand hybridizes with a nucleic acid molecule from a) or b) under stringent conditions and which exhibit the biological function of a photoprotein:
(d) nucleic acid molecules which differ from the nucleic acid molecules mentioned under c) due to the degeneracy of the genetic code;
(e) nucleic acid molecules which exhibit a sequence homology with SEQ ID NO: 1 of at least 95% and have the biological function of a photoprotein; and
(f) nucleic acid molecules which exhibit a sequence homology with SEQ ID NO: 1 of at least 65% and have the biological function of a photoprotein.

7. A method for expressing a polypeptide comprising:
expressing in a bacterium, a eukaryotic cell, or an in vitro expression system a nucleic acid molecule encoding the photoprotein polypeptide of claim 6.

8. (canceled)

9. A peptide having more than 5 consecutive amino acids which is recognized immunologically by antibodies directed against the photoprotein berovin.

10-11. (canceled)


SEQUENCE LISTING (2 sequences)

SEQ ID NO: 1 - DNA, 1160 bp, Beroe abyssicola
gagttttaaactttatacaacactactttataaaatctaatttgagccaatcaaaatgactgaacgtctgaacgagcagaacaacgagagttaccgctacctgagaagcgtgggaaaccagtggcagttcaacgtagaggacctccaccccaagatgttgtcccgtctctacaagagattcgatactttcgatctagacagtgacggtaagatggagatggacgaggtcttgtactggcccgacaggatgaggcagctggtaaacgctactgatgagcaggttgagaagatgcgggatgctgtgagagttttctttttgcacaagggagtggagccagtaaacggtctcctcagagaggactgggtggaagctaacagagtcttcgctgaggctgagagagaaagagagcgacgaggagaaccttctcttatcgcacttctctccaactcttactacgatgtactggatgatgacggtgatggtactgttgacgtcgatgaattaaagaccatgatgaaagcatttgatgtgccccaggaagctgcctacaccttcttcgagaaggcagacactgacaagagtggaaagttggagagaacagaactagttcatctctttagaaagttttggatggagccttacgatccacagtgggacggagtctacgcttataagtactaataaattatcggatccagaacaaacggcaagaactattttacactccacttcaattataaacgatgatttcatcgtttcatgaaaacaaattttagcataaaacaataaacattttcgactactaaaattacagtcaacataaaaattttaaagtgtgtgataaatttttctgaatgtctcctaacttcgataataatcttcaaacttttagtcaatatatggaaataaaaatattttggtttgatgagatgataaaacttttgttttaaattttagttggagtaattcattgaatatgaaatggacttcggacaattaccctggccttctgtatatttaaagcatgctttccgtcaataatttgttgtatctatgtatttatttgtacattaatttaaccatatagaattatctgtataatccctgttatatttatacactgcattacaaaaaaaaaaaaaaaaaaaaaaaaaaaaaa

SEQ ID NO: 2 - protein, 208 aa, Beroe abyssicola
MTERLNEQNNESYRYLRSVGNQWQFNVEDLHPKMLSRLYKRFDTFDLDSDGKMEMDEVLYWPDRMRQLVNATDEQVEKMRDAVRVFFLHKGVEPVNGLLREDWVEANRVFAEAERERERRGEPSLIALLSNSYYDVLDDDGDGTVDVDELKTMMKAFDVPQEAAYTFFEKADTDKSGKLERTELVHLFRKFWMEPYDPQWDGVYAYKY